(19) United States
(12) Patent Application Publication
(10) Pub. No.: US 2026/0232303 A1
(43) Pub. Date: Aug. 13, 2026

(54) IRRIGATION OUTLFOW CONTROL

(71) Applicant: Auris Health, Inc., Santa Clara, CA (US)

(72) Inventors: Elizabeth Abigail Eaton, Redwood City, CA (US); Chiarra Gatti, San Francisco, CA (US); Grace Magdalena Restle, Santa Clara, CA (US)

(73) Assignee: Auris Health, Inc., Santa Clara, CA (US)

(21) Appl. No.: 19/052,179

(22) Filed: Feb. 12, 2025

(51) Int. Cl. A61B 17/00; A61M 1/00

(52) U.S. Cl. CPC A61B 17/00234; A61M 1/74; A61B 2017/00862; A61B 2217/005; A61M 2202/0014; A61M 2202/04; A61M 2205/0216; A61M 2205/3331

(57) ABSTRACT

This disclosure provides a medical system for percutaneous access procedures. The present implementations more specifically relate to a fluid outflow control device that can be used to control a flow of fluid exiting an anatomy along an outer surface of a percutaneous access instrument. In some aspects, the fluid outflow control device may be placed around the outer surface of the percutaneous access instrument to impede the flow of fluid along the outer surface and divert the fluid into a collection apparatus. In some implementations, the fluid outflow control device may include a flow impeding element that can form a seal around the outer surface of the instrument that prevents the fluid from flowing any further along the outer surface and redirects the fluid into a fluid diversion element that can deposit the fluid securely into the collection apparatus.

### TECHNICAL FIELD

This disclosure relates generally to medical systems, and specifically to irrigation outflow control for medical systems.

### DESCRIPTION OF RELATED ART

Many medical procedures, such as laparoscopy, ureteroscopy, or percutaneous nephrolithotomy (PCNL), involve a series of complex steps that require careful movement and positioning of medical tools or instruments inside a patient's body. For example, to remove urinary stones from the kidney and ureter, a physician can insert a ureteroscope into the urinary tract through the urethra. A ureteroscope includes an endoscope at its distal end configured to enable visualization of the urinary tract. Generally, during a percutaneous access procedure (such as PCNL), the physician (or a technician) drives a needle into the patient, through a target location on the kidney, and uses another medical instrument (which may be in conjunction with the needle) to extract the stone from the kidney via the percutaneous access point.

Some medical procedures utilize saline fluids for irrigation and aspiration (also referred to herein as “fluidics”). As used herein, the term “irrigation” refers to the movement or delivery of fluid into an anatomy and the term “aspiration” refers to the movement or extraction of fluid out of the anatomy. Example forms of aspiration can include active suction and/or passive outflow of fluid. Fluidics can be used for various purposes such as, for example, to achieve distension of the anatomy (such as for endoscopic vision), maintain suitable intrarenal pressures during the procedure (such as to prevent damage to the anatomy), or move around objects (such as urinary stones) within the anatomy. Thus, the flow rates and/or pressures associated with irrigation and aspiration can affect various aspects of a medical procedure.

In particular, fluid outflow is needed to prevent over-pressurization of the anatomy, which can result in tissue damage, and under-pressurization of the anatomy, which can result in insufficient anatomical distention that is needed for visualization, manipulating instruments, and/or removing objects (such as stone fragments) from the anatomy. In some instances, fluid flowing out of the anatomy can adhere to the outer surface of a percutaneous access instrument (such as a suction catheter). Depending on various factors (such as the volume of fluid entering and/or exiting the anatomy, the characteristics of the anatomical opening, and the angle and/or depth of insertion of the percutaneous access instrument), the fluid may flow any distance along the outer surface of the instrument before dropping off from the instrument due to gravity. The falling waste fluid can pool or accumulate on the floor and/or other nearby surfaces, creating a hazardous environment for the physician and staff.

Existing fluid waste collection solutions include placing a container or pouch below the percutaneous access instrument to catch the fluid that drops from the outer surface of the instrument. Because the waste fluid can drop from varying points along the length of the instrument, such containers often have a relatively large footprint which can compete for space in an already-crowded operating environment. Moreover, a physician may still need to move or reposition the container in response to changes in the outflow of waste fluid. Thus, there is a need to control the outflow of fluid on the outer surface of a percutaneous access instrument to prevent the fluid from pooling or accumulating on undesired surfaces.

### SUMMARY

This Summary is provided to introduce in a simplified form a selection of concepts that are further described below in the Detailed Description. This Summary is not intended to identify key features or essential features of the claimed subject matter, nor is it intended to limit the scope of the claimed subject matter.

One innovative aspect of the subject matter of this disclosure can be implemented in a medical system including a medical instrument, a waste collection apparatus, and an outflow control device. The medical instrument has an elongate shaft configured to be inserted percutaneously into an anatomy. The waste collection apparatus is configured to capture fluid that flows out of the anatomy along an outer surface of the elongate shaft. The outflow control device is configured to impede the flow of fluid along the outer surface of the elongate shaft and divert the fluid to the waste collection element.

Another innovative aspect of the subject matter of this disclosure can be implemented in a fluid outflow control device for a medical instrument having an elongate shaft. The fluid outflow control device includes a flow impeding element, a fluid diversion element, and an interface element. The flow impeding element is configured to form a seal around an outer surface of the elongate shaft that redirects a flow of fluid along the outer surface onto a surface of the flow impeding element. As used herein, the term “seal” refers to an impediment that slows or stops the flow of fluid along the outer surface of the shaft. Thus, the seal need not be airtight or watertight to achieve the benefits of the present disclosure. The fluid diversion element is configured to form a channel for carrying the fluid between the flow impeding element and a waste collection apparatus. The interface element is configured to funnel the fluid from the surface of the flow impeding element into the fluid diversion element.

### BRIEF DESCRIPTION OF THE DRAWINGS

The present implementations are illustrated by way of example and are not intended to be limited by the figures of the accompanying drawings.

FIG. 1 shows an example medical system, according to some implementations.

FIG. 2 shows an example medical procedure involving fluidics, according to some implementations.

FIG. 3 shows an example medical system including a fluid outflow control device, according to some implementations.

FIGS. 4A-4C show an example flow impeding element for a fluid outflow control device, according to some implementations.

FIGS. 5A and 5B show another example flow impeding element for a fluid outflow control device, according to some implementations.

FIG. 6 shows an example fluid diversion element for a fluid outflow control device, according to some implementations.

### DETAILED DESCRIPTION

In the following description, numerous specific details are set forth such as examples of specific components, circuits, and processes to provide a thorough understanding of the present disclosure. The term “coupled” as used herein means connected directly to or connected through one or more intervening components or circuits. Also, in the following description and for purposes of explanation, specific nomenclature is set forth to provide a thorough understanding of the aspects of the disclosure. However, it will be apparent to one skilled in the art that these specific details may not be required to practice the example implementations. In other instances, well-known circuits and devices are shown in block diagram form to avoid obscuring the present disclosure.

Certain standard anatomical terms of location may be used herein to refer to the anatomy of animals, and namely humans, with respect to the example implementations. Although certain spatially relative terms, such as “outer,” “inner,” “upper,” “lower,” “below,” “above,” “vertical,” “horizontal,” “top,” “bottom,” and similar terms, are used herein to describe a spatial relationship of one element, device, or anatomical structure to another device, element, or anatomical structure, it is understood that these terms are used herein for ease of description to describe the positional relationship between elements and structures, as illustrated in the drawings. It should be understood that spatially relative terms are intended to encompass different orientations of the elements or structures, in use or operation, in addition to the orientations depicted in the drawings. For example, an element or structure described as “above” another element or structure may represent a position that is below or beside such other element or structure with respect to alternate orientations of the subject patient, element, or structure, and vice-versa. As used herein, the term “patient” may generally refer to humans, anatomical models, simulators, cadavers, and other living or non-living objects.

As described above, some medical procedures utilize saline fluids for irrigation and aspiration (also referred to herein as “fluidics”). As used herein, the term “irrigation” refers to the movement or delivery of fluid into an anatomy and the term “aspiration” refers to the movement or extraction of fluid out of the anatomy. Fluidics can be used for various purposes such as, for example, to achieve distension of the anatomy (such as for endoscopic vision), maintain suitable intrarenal pressures during the procedure (such as to prevent damage to the anatomy), or move around objects (such as urinary stones) within the anatomy. Thus, the flow rates and/or pressures associated with irrigation and aspiration can affect various aspects of a medical procedure.

In particular, fluid outflow is needed to prevent over-pressurization of the anatomy, which can result in fractures, tissue breakage, or damage to the anatomy. In some instances, fluid flowing out of the anatomy can adhere to the outer surface of a percutaneous access instrument (such as a suction catheter). Depending on various factors (such as the volume of fluid entering and/or exiting the anatomy, the characteristics of the anatomical opening, and the angle and/or depth of insertion of the percutaneous access instrument), the fluid may flow any distance along the outer surface of the instrument before dropping off from the instrument due to gravity. The falling waste fluid can pool or accumulate on the floor and/or other nearby surfaces, creating a hazardous environment for the physician and staff.

In some aspects, a fluid outflow control device may be placed around the outer surface of the percutaneous access instrument to impede the flow of fluid along the outer surface and divert the fluid into a waste collection apparatus. In some implementations, the fluid outflow control device may include a flow impeding element that can form a seal around the outer surface of the instrument that prevents the fluid from flowing any further along the outer surface and redirects the fluid into a fluid diversion element that can deposit the fluid securely into a waste collection apparatus. As used herein, the term “seal” refers to an impediment that slows or stops the flow of fluid along the outer surface of the shaft. Thus, the seal need not be airtight or watertight to achieve the benefits of the present disclosure. In some implementations, the fluid outflow control device may be formed from multiple discrete components that can be attached or coupled to one another (such as in a modular fashion). In some other implementations, two or more components of the fluid outflow control device may be integrated with one another to form a continuous object.

The flow impeding element can be coupled to the percutaneous access instrument at any point along the length of its outer surface to capture any fluid that may flow along the outer surface before it would otherwise fall due to gravity. In other words, the flow impeding element can be positioned close enough to the patient so that at least some of the fluid exiting the anatomy will at least flow onto a surface of the flow impeding element. Further, the flow impeding element redirects the waste fluid into the fluid diversion element, which can carry the fluid away from the instrument with little or no leakage or spilling (such as within an enclosed channel) and deposit the fluid in a waste collection apparatus that can be placed at any suitable location within (or outside) the operating environment. In some implementations, the fluid outflow control device may further include an interface element to funnel the fluid from the flow impeding element into the fluid diversion element without spilling any of the fluid onto the floor or other surfaces.

Particular implementations of the subject matter described in this disclosure can be implemented to realize one or more of the following potential advantages. The fluid outflow control device of the present disclosure can control the flow of fluid exiting an anatomy along the outer surface of a percutaneous access instrument to prevent such waste fluid from pooling or accumulating on the floor and/or other undesired surfaces. More specifically, the fluid outflow control device can ensure that all waste fluid is properly diverted to and captured by a waste collection or management device. This allows a user to more accurately measure or otherwise assess the volume of fluid removed from the anatomy (such as by inspecting the contents of the waste fluid). Compared to existing solutions, the fluid outflow control device of the present disclosure can have a significantly smaller footprint while achieving greater efficacy at capturing and collecting waste fluids. Further, the fluid outflow control device can be constructed or manufactured using relatively inexpensive materials and can be coupled or attached to a percutaneous access instrument (and waste collection device) without adding much complexity to existing workflows for setting up and performing percutaneous access procedures.

Aspects of the present disclosure may be used to perform robotic-assisted medical procedures, such as endoscopic access, percutaneous access, or treatment for a target anatomical site. For example, robotic tools may engage or control one or more medical instruments (such as an endoscope and/or a percutaneous access catheter) to access a target site within a patient's anatomy or perform a treatment at the target site. In some implementations, the robotic tools may be guided or controlled by a physician. In some other implementations, the robotic tools may operate in an autonomous or semi-autonomous manner. Although systems and techniques are described herein in the context of robotic-assisted medical procedures, the systems and techniques may be applicable to other types of medical procedures (such as procedures that do not rely on robotic tools or only utilize robotic tools in a very limited capacity). For example, the systems and techniques described herein may be applicable to medical procedures that rely on manually operated medical instruments (such as a percutaneous access catheter that is exclusively controlled and operated by a physician). The systems and techniques described herein also may be applicable beyond the context of medical procedures (such as in simulated environments or laboratory settings, such as with models or simulators, among other examples).

Although certain aspects of the present disclosure are described in detail herein in the context of urological procedures, such as kidney stone removal and treatment procedures, it should be understood that such context is provided for convenience and clarity, and the concepts disclosed herein are applicable to any suitable medical procedure. However, as mentioned, description of the urinary anatomy and associated medical issues and procedures is presented herein to aid in the description of the concepts disclosed herein. In some implementations, the techniques and systems described herein are discussed in the context of a percutaneous procedure, which can include any procedure where access is gained to a target location by making a puncture or incision in the skin, mucous membrane, or other body layer. However, it should be understood that these techniques and systems can be implemented in the context of any medical procedure involving irrigation and/or aspiration of any type of fluid (such as saline solutions and/or blood) via a percutaneous access instrument.

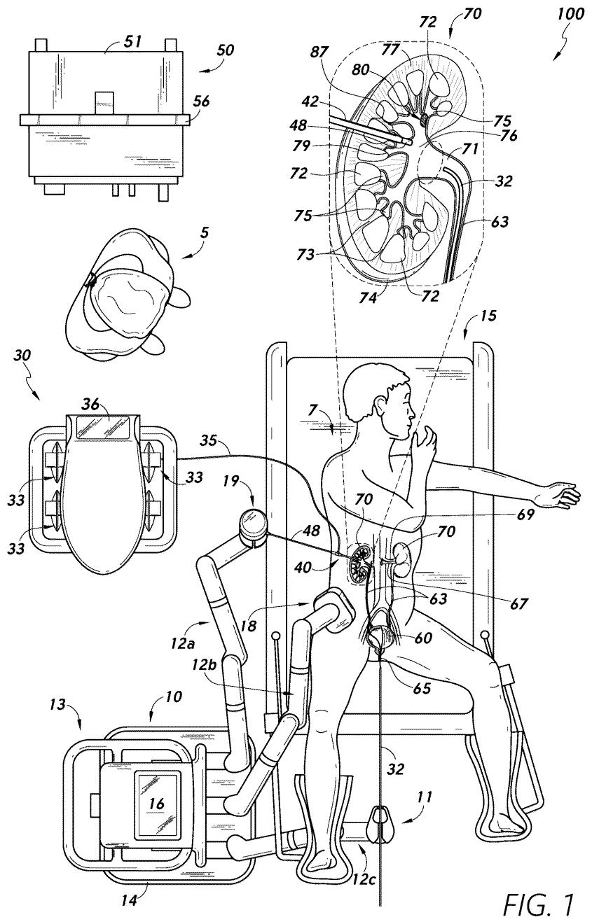

FIG. 1 shows an example medical system 100, according to some implementations. In some aspects, the medical system 100 may be used for percutaneous and/or endoscopic (such as ureteroscopic) medical procedures. As referenced and described above, certain ureteroscopic procedures involve the treatment or removal of kidney stones. In some implementations, kidney stone treatment can benefit from the assistance of certain robotic technologies or devices, such as may be similar to those shown in FIG. 1 and described in detail below. Robotic medical solutions can provide relatively higher precision, superior control, and/or superior hand-eye coordination with respect to certain instruments compared to strictly-manual procedures. For example, robotic-assisted percutaneous access to the kidney in accordance with some procedures can advantageously enable a urologist to perform both direct-entry endoscopic renal access and percutaneous renal access.

In certain stone management procedures, fluid irrigation may be implemented in order to maintain desired kidney distention, which may advantageously facilitate visualization and/or navigation within the target treatment site (such as a calyx network of a kidney). However, it may be desirable or necessary to control fluid irrigation at least in part to avoid over-pressurizing the kidney, which can result in physiological harm to the patient and/or damage to the renal anatomy. Specifically, with respect to renal procedures, over-pressurization can result in fractures, tissue breakage, and/or other physical damage. Fluid from one area, such as the kidney, may escape into inappropriate areas through tissue damage or rupture, which can lead to potential sequelae (such as sepsis) through existing infectious material, through stagnation or breakdown of trapped material, or through exposure of tissues not normally exposed to such fluid. For example, intrarenal infection may result from the presence of one or more kidney stones. Infected intrarenal fluid that is expelled or otherwise passes into the bloodstream as a result of damage from over-pressurization can result in complications as described above. Therefore, it may be desirable to limit irrigation pressure levels in order to promote the desired or sufficient kidney distention to perform a stone management procedure without causing undesirably high intrarenal pressures. Furthermore, under-pressurization can result in the lack of effective anatomical distention for visualization, which can reduce the efficacy of a procedure and/or result in damage to the internal anatomy.

The medical system 100 includes a robotic system 10 (such as a mobile robotic cart) configured to engage with and/or control a medical instrument 32 (such as a ureteroscope) to perform a direct-entry procedure on a patient 7. The term “direct entry” is used herein according to its broad and ordinary meaning and may refer to any entry of instrumentation through a natural or artificial opening in a patient's body. For example, with reference to FIG. 1, the direct entry of the scope 32 into the urinary tract of the patient 7 may be made via the urethra 65.

In the illustrated system 100, a percutaneous access instrument 40 is further implemented to provide percutaneous access to the kidney 70. The percutaneous access instrument 40 may include one or more sheaths and/or shafts through which instruments and/or fluids may access the target anatomy in which the distal end of the instrument 40 is disposed. The term “percutaneous access” is used herein according to its broad and ordinary meaning and may refer to entry, such as by puncture and/or incision, of instrumentation through the skin of a patient and any other body layers necessary to reach a target anatomical location associated with a procedure (such as the calyx network of the kidney 70). The term “percutaneous access instrument” is used herein according to its broad and ordinary meaning and may refer to a surgical tool, device, or assembly that is configured to puncture or to be inserted through skin and/or other tissue or anatomy, such as a needle, a scalpel, a guidewire, sheath, shaft, scope, dilators, and the like. However, it should be understood that a percutaneous access instrument can refer to other types of medical instruments in the context of the present disclosure. It should be understood that the percutaneous access instruments 40, 48 and direct-entry instrument 32 may be any type of medical instruments, including endoscopes (such as a ureteroscope), catheters (such as a steerable or non-steerable catheter), a nephroscopes, laparoscopes, or other type of medical instrument.

The system 100 may include a catheter 48, which may access the internal renal anatomy through the percutaneous access instrument 40. In some implementations, the catheter 48 may be manipulated and/or held in place by a tool or coupling 19 coupled to an arm 12a of the robotic system 10. The catheter 48 may be a flexible, robotically-driven instrument. In some implementations, an irrigation backflow channel may be formed in the space between the outer wall of the catheter 48 and an inner wall or sheath of the percutaneous access device or assembly 40, wherein the catheter 48 is disposed within a channel formed by such inner wall or sheath. With the catheter 48 disposed within the percutaneous access instrument 40, the catheter 48 and the shaft(s) or sheath(s) of the percutaneous access instrument 40 may be generally concentric. The catheter 48 and the percutaneous access instrument 40 may advantageously have generally circular cross-sectional shape over at least a portion thereof.

In some implementations, the percutaneous access instrument or assembly 40 and/or other medical instruments of the system 100 form or provide multiple passive fluid outflow channels. For example, passive outflow channels may include a channel formed between the outer wall of the scope 32 and an access sheath through which the scope 32 is passed or disposed. As another example, a working channel of the scope 32 may provide a passive irrigation backflow path from the kidney 70. In some implementations, active outflow is provided through the percutaneous catheter 48 (such as active suction). In cases in which active suction is not implemented within the percutaneous catheter 48, passive irrigation backflow may flow therethrough to some degree. In some configurations, the greatest volume of passive irrigation backflow may be between the outside of the catheter 48 and the inner wall or sheath of the percutaneous access instrument or assembly 40.

The medical system 100 also includes a fluid management cart 30, which may be configured to hold one or more fluid bags or containers 33 and/or control fluid flow therefrom. For example, an irrigation fluid line 35 (also referred to as an “irrigation line” or a “fluid line”) may be coupled to one or more of the bags or containers 33 and to an irrigation port of the percutaneous access instrument or assembly 40. Irrigation fluid may be provided to the target anatomy via the irrigation line 35 and the percutaneous access instrument or assembly 40. The fluid management cart 30 may include certain electronic components, such as a display 36, flow control mechanics, and/or certain associated control circuitry. The fluid management cart 30 may have one or more IV bags 33 hanging on one or more sides thereof. The cart 30 may include one or more pumps with which aspiration fluid may be pulled into a collection container or cartridge. In some implementations, the irrigation fluid pressure may be determined at least in part with respect to one or more points along the irrigation and/or aspiration fluid channel(s).

The medical system 100 also includes a control system 50 configured to interface with the robotic system 10 and/or fluid cart 30, provide information regarding the procedure, and/or perform a variety of other operations. For example, the control system 50 can include one or more display(s) 56 configured to present certain information to assist the physician 5 and/or other technician(s) or individual(s). The medical system 10 can include a table 15 configured to hold the patient 7. The system 10 may further include an electromagnetic (EM) field generator 18, which may be held by one or more of the robotic arms 12 of the robotic system 10 or may be a stand-alone device. Although the various robotic arms are shown in various positions and coupled to various instrumentation, it should be understood that such configurations are shown for convenience and illustration purposes, and such robotic arms may have different configurations over time and/or at different points during a medical procedure. In some implementations, the arm 12a is configured to hold or control the catheter 48 only after removing the electromagnetic field generator 18 therefrom. That is, the instrument coupling 19 and the field generator 18 may generally be mounted to the same robotic arm as interchanged over time.

In some implementations, the system 100 may be used to perform a percutaneous procedure, such as percutaneous nephrolithotomy (PCNL). To illustrate, if the patient 7 has a kidney stone 80 that is too large to be removed or passed through the urinary tract (63, 60, 65), the physician 5 can perform a procedure to remove the kidney stone 80 through a percutaneous access point or path associated with the flank or side of the patient 7. In some implementations, the physician 5 can interact with the control system 50 and/or the robotic system 10 to cause or control the robotic system 10 to advance and navigate the medical instrument 32 (such as a scope) from the urethra 65, through the bladder 60, up the ureter 63, and into the calyx network of the kidney 70 where the stone 80 is located. The physician 5 can further interact with the control system 50 and/or the robotic system 10 to cause or control the advancement of the catheter 48 through the percutaneous access instrument 40. The control system 50 can provide information via the display(s) 56 that is associated with the medical instrument 32, such as real-time endoscopic images captured therewith, and/or other instruments of the system 100, to assist the physician 5 in navigating or controlling such instrumentation.

The renal anatomy is described herein for reference with respect to certain medical procedures relating to aspects of the present disclosure. The kidneys 70, shown roughly in typical anatomical position in FIG. 1, generally comprise two bean-shaped organs located on the left and right sides, respectively, in the retroperitoneal space. However, congenital variations can cause some people to have one or three kidneys, a horseshoe kidney, multiple renal arteries and/or veins, or duplicate ureters, among other examples. In adult humans, the kidneys are generally about 11 cm in height or length. The kidneys receive blood from the paired renal arteries 69; blood exits the kidney via the paired renal veins 67. Each kidney 70 is fluidly coupled with a respective ureter 63, which generally comprises a tube that carries excreted urine from the kidney 70 to the bladder 60.

A recessed area on the concave border of the kidney 70 is the renal hilum 78, where the renal artery (not shown in the detailed view of the kidney 70) enters the kidney 70 and the renal vein (not shown in detailed view) and ureter 63 leave. The kidney 70 is surrounded by tough fibrous tissue, the renal capsule 74, which is itself surrounded by perirenal fat, renal fascia, and pararenal fat. The anterior (front) surface of these tissues is the peritoneum, while the posterior (rear) surface is the transversalis fascia.

The functional substance, or parenchyma, of the kidney 70 is divided into two major structures: the outer renal cortex 77 and the inner renal medulla 87. These structures take the shape of a plurality of generally cone-shaped renal lobes, each containing renal cortex surrounding a portion of medulla called a renal pyramid 72. Between the renal pyramids 72 are projections of cortex called renal columns 73. Nephrons (not shown in detail in FIG. 1), the urine-producing functional structures of the kidney, span the cortex 77 and medulla 87. The initial filtering portion of a nephron is the renal corpuscle, which is located in the cortex and is followed by a renal tubule that passes from the cortex deep into the medullary pyramids. Part of the renal cortex, a medullary ray, is a collection of renal tubules that drain into a single collecting duct.

The tip or apex, or papilla 79, of each renal pyramid empties urine into a respective minor calyx 75; minor calyces 75 empty into major calyces 76, and major calyces 76 empty into the renal pelvis 71, which transitions to the ureter 63. The manifold type collection of minor and major calyces may be referred to herein as the “calyx network” of the kidney. At the hilum 78, the ureter 63 and renal vein exit the kidney and the renal artery enters. Hilar fat and lymphatic tissue with lymph nodes surrounds these structures. The hilar fat is contiguous with a fat-filled cavity called the renal sinus. The renal sinus collectively contains the renal pelvis 71 and calyces 75, 76 and separates these structures from the renal medullary tissue. The funnel or tubular-shaped anatomy associated with the calyces can be referred to as the infundibulum or infundibula. That is, an infundibulum generally leads to the termination of a calyx where a papilla is exposed within the calyx.

With further reference to the medical system 100, the medical instrument 32 (such as a scope, directly-entry instrument, etc.) can be advanced into the kidney 70 through the urinary tract. Once at the site of the kidney stone 80 (such as within a target calyx 75 of the kidney 70 through which the stone 80 is accessible), the medical instrument 32 can be used to designate or tag a target location for percutaneous access to the kidney 70. To minimize damage to the kidney and/or surrounding anatomy, the physician 5 can designate a particular papilla 79 of the kidney 70 as the target location or anatomical feature for entering into the kidney 70 with a percutaneous access instrument (such as a needle). However, other target locations can be designated or determined. Once the percutaneous access instrument(s) has reached the target location (such as the calyx 75), the utilized percutaneous access path may be used to extract the kidney stone 80 from the patient 7.

Fluid may be directed into the calyx network using the fluid cart 30 and irrigation line 35 throughout at least portions of the procedure to produce desirable kidney distension for navigation and viewing. In cases of under-pressurization, wherein there is not enough fluid in the kidney to produce desired or necessary distension, medical instruments can damage or unintentionally puncture parts of the kidney. For example, fluid is an important medium for laser lithotripsy, as cavitation created by the laser beam can help break up kidney stones. Further, laser lithotripsy can result in accidental damage to tissue at the treatment site by the laser as a result of the collapse of the surrounding anatomy from under-pressurization.

In the example of FIG. 1, the medical instrument 32 is implemented as a scope. However, in some other implementations, the medical instrument 32 may be implemented as any suitable type of medical instrument, such as scope, a catheter, a guidewire, a lithotripter, a basket retrieval device, and so on. In some implementations, the medical instrument 32 may be a steerable device.

The various scope-type instruments disclosed herein, such as the scope 32 of the system 100, can be configured to navigate within the human anatomy, such as within a natural orifice or lumen of the human anatomy. The terms “scope” and “endoscope” are used herein according to their broad and ordinary meanings, and may refer to any type of elongate medical instrument having image generating, viewing, and/or capturing functionality and configured to be introduced into any type of organ, cavity, lumen, chamber, or space of a body. A scope can refer to, for example, a ureteroscope (such as for accessing the urinary tract), a laparoscope, a nephroscope (such as for accessing the kidneys), a bronchoscope (such as for accessing an airway, such as the bronchus), a colonoscope (such as for accessing the colon), an arthroscope (such as for accessing a joint), a cystoscope (such as for accessing the bladder), borescope, and so on. Scopes or endoscopes, in some instances, may comprise a rigid or flexible tube, and may be dimensioned to be passed within an outer sheath, catheter, introducer, or other lumen-type device, or may be used without such devices. Some scopes or endoscopes can also have their own working channels or lumens.

Irrigation fluid may be provided to the treatment site (such as the kidney 70) through the percutaneous access device 40, through the percutaneous access catheter 48, and/or through the direct-entry device 32. Furthermore, irrigation and aspiration may or may not be provided through the same instrument(s). Where one or more of the instruments (32, 40, 48) provides the irrigation and/or aspiration functionality, one or more others of the instruments may be used for other functionality, such as breaking-up the object 80 to be removed.

The control system 50 can be configured to provide various functionality to assist in performing a medical procedure. In some implementations, the control system 50 can be coupled to the robotic system 10 and/or fluid management system 30 and operate in cooperation therewith to perform a medical procedure on the patient 7. For example, the control system 50 can communicate with the robotic system 10 and/or fluid management system 30 via a wireless or wired connection (such as to control the robotic system 10, fluid flow from the fluid management system 30, etc.). Further, in some implementations, the control system 50 can communicate with a needle and/or nephroscope to receive position data therefrom. Moreover, in some implementations, the control system 50 can communicate with the table 15 to position the table 15 in a particular orientation or otherwise control the table 15. In some implementations, the control system 50 can communicate with the EM field generator 18 to control generation of an EM field in an area around the patient 7.

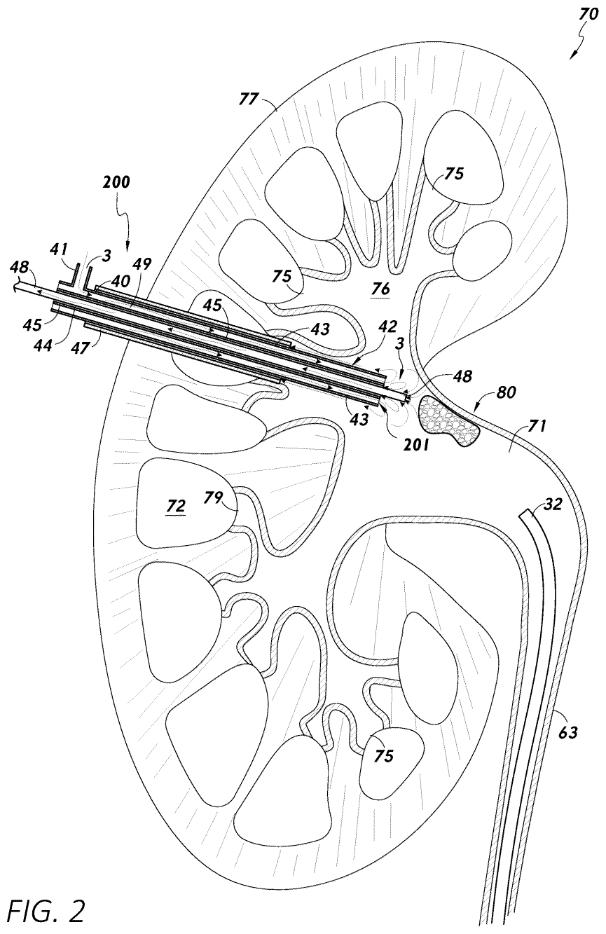

FIG. 2 shows an example medical procedure using fluidics, according to some implementations. For example, a device or system similar to the percutaneous access device or assembly 40 may be used to provide irrigation (inflow) to a treatment site, such as an internal calyx network of a kidney 70. Fluid irrigation and aspiration (generally referred to as “fluidics” herein) can represent an important component of certain medical procedures. For example, during a PCNL procedure, fluidics may be applied to distend the kidney 70 and/or clear stone dust, small fragments, and thrombus from the treatment site as well as the visual field provided by the medical instrument(s). For example, with respect to the implementation of FIG. 2, irrigation fluid 3 can be provided through a channel 49 of the percutaneous access instrument 40. Aspiration (outflow) may exit the treatment site through one or more passive and/or active outflow channels, which may or may not be associated with the percutaneous access instrument 40. In some implementations, irrigation and aspiration can both be active.

In some aspects, the medical procedure depicted by FIG. 2 may be a PCNL procedure. The illustrated renal anatomy includes an object disposed in the calyx network of the kidney 70, wherein the object 80 can be any object that is targeted for removal, such as a kidney stone. In the illustrated example, the medical instrument assembly 200 includes a percutaneous access laparoscope or percutaneous access instrument 40. The percutaneous access instrument 40 can be inserted percutaneously into the kidney 70 through an access sheath 47. According to some implementations, the access sheath 47 may be placed by first accessing the treatment site with a rigid needle and using a dilator to dilate the percutaneous access path and place the sheath 47. The percutaneous access instrument 40 can include a working channel 44 within an inner shaft or wall 45, though which various tools can be inserted, such as a catheter 48. In some implementations, a lithotripter (such as an ultrasonic lithotripter) may be inserted through the working channel 44 of the percutaneous access instrument 40. The percutaneous access instrument 40 can also include an optic device (not shown) configured to allow a surgeon to visualize the treatment site.

The catheter 48 may be navigated within the kidney 70 by torquing the catheter 48 and/or percutaneous access instrument 40 towards the object 80. In some implementations, the object or stone 80 may be broken-up using a lithotripter (not shown) and removed in smaller fragments through the percutaneous access catheter 48. The lithotripter may be advanced to the treatment site through percutaneous or direct entry (such as through the percutaneous access instrument 40 or through a sheath through which the scope 32 is advanced).

As illustrated with arrows in FIG. 2, irrigation (such as a saline solution) can be applied to the treatment site (such as the kidney 70) through the percutaneous access instrument 40. The irrigation fluid 3 may enter the percutaneous access instrument 40 through an irrigation port 41 and exit through a distal end 201 into the kidney 70. Irrigation can be used to clear stone dust and small fragments from the field of view of, for example, the scope 32 or other image or viewing device to allow the surgeon to visualize the treatment site, as well as to distend the kidney 70 to allow access to the object 80. In the illustrated example, aspiration is also applied to the treatment site through the medical instrument 40. As shown, fluid can be removed from the kidney 70 through the access sheath 47 (such as between the outer shaft 43 of the percutaneous access instrument 40 and the sheath 47) and/or through the working channel 44 of the percutaneous access instrument 40 (such as between the inner shaft 45 of the percutaneous access instrument 40 and the catheter 48). In some implementations, aspiration may be provided through a channel in a lithotripter. In some instances, aspiration is pulled (actively) through one or more outflow channels and/or permitted to passively flow through one or more outflow channels. For example, active aspiration or suction may be drawn through the catheter 48. In some implementations, fluidics are applied during substantially the entire procedure.

The fluidics applied during the procedure can establish a fluid flow as illustrated by the arrows in FIG. 2. Initially, fluid can flow outward from the distal tip 201 of the percutaneous access instrument 40 towards the object 80. Aspiration through the access sheath 47 and/or working channel 44 can cause fluid flow back towards the percutaneous access instrument or sheath. As illustrated, in the region of the object 80, the flow may be both directed toward and away from the object 80 with respect to the distal end 201 of the percutaneous access instrument 40. Where the available fluid outflow channels are insufficient to remove a flow of fluid equal to the irrigation flow into the treatment site, risks of over-filling the kidney can be present.

During a ureteroscopic lithotripsy procedure, the ureteroscope 32 may enter the kidney 70 through the ureter 63 and use stone-retrieval basket(s) and/or lithotripter(s) to relocate and break down kidney stones, respectively. For example, a lithotripter can be deployed through a working channel of the ureteroscope 32 and used to break the stone 80 into fragments, which may be aspirated through the catheter 48.

Irrigation and/or aspiration can be managed to produce desirable flow characteristics resulting in desirable distension conditions for the target organ or anatomy. In some implementations, irrigation (inflow) enters the treatment site through a first medical device (such as the percutaneous access instrument 40 and/or catheter 48 disposed therein). In some implementations, the percutaneous access instrument 40 and/or catheter 48 can be inserted into the treatment site antegrade of an object (such as a kidney stone) 80 to be removed, whereas another medical instrument (such as an endoscope) 32 can be inserted into the treatment site retrograde of the object 80. Although FIG. 2 shows the irrigation fluid 3 as provided to the treatment site percutaneously, in some other implementations, irrigation may be provided via a medical instrument that accesses the treatment site through direct entry (such as the ureteroscope 32). Any of the percutaneous access instruments 40, 48 or direct-entry instruments 32 may be robotically controlled as described above with reference to FIG. 1. Accordingly, aspects of the present disclosure can be employed robotically in some implementations.

In some implementations, the percutaneous access instrument 40 may provide irrigation with a sufficiently high inflow rate without causing turbulence. This may allow the treatment site (such as the kidney 70) to fill up with fluid without displacing the object 80. In some other implementations, the irrigation and/or aspiration rate(s) can be modulated to improve stone displacement or stabilization or to intentionally create turbulence so that the irrigation reaches all corners of the treatment site. For example, a gentle alternating cycle of irrigation and aspiration can create a lavage-like effect to preferentially pull large stone debris away from calyces and towards the aspiration site(s). Alternatively, short pulsatile inflow and outflow could be used to create turbulence and ensure that smaller and lighter stone fragments do not settle on the floor of the treatment site, but instead remain floating in the irrigation fluid and eventually are aspirated with the outflow.

The percutaneous access catheter 48 can be an articulable catheter that is introduced via percutaneous access into the treatment site (such as the calyx network of the kidney 70). The catheter 48 can be navigated within the kidney 70. For example, the catheter 48 may be configured to be inserted and retracted into the treatment site and/or to articulate (such as to bend) therein. In some implementations, the catheter 48 can include pull-wires for controlling articulation. For example, four pull-wires may be oriented in the four orthogonal directions to enable articulation of the catheter 48. Other methods for permitting articulation of the catheter are also possible. The catheter 48 can include, for example, an aspiration lumen (or channel). The aspiration lumen can be fluidly coupled to a pump or vacuum device (such as an external pump). The pump or vacuum may generate negative pressure that causes flow from the treatment site into the catheter. The aspiration function may be able to be toggled (such as on and off) and adjusted by the user or system. In some implementations, the aspiration lumen may be used for irrigation as well.

The catheter 48 can provide various functions during an object removal procedure, such as stone stabilization during lithotripsy. For example, if the stone 80 is larger than the aspiration lumen of the catheter, the stone can be held at the distal face of the aspiration lumen, thus stabilizing the stone while it is broken down to dust and smaller fragments. Active aspiration may hold the stone to the distal face of the catheter 48. In some cases, a stone being extracted can substantially seal off the catheter 48, thereby causing the stone to be held by the catheter due to the pressure differential. This may provide the user with a less-mobile target for lithotripsy. Moreover, the catheter 48 can improve visibility of the treatment site by removing stone dust from the kidney. This can provide the user with improved visibility (such as continuously adequate visibility), for example, from an imaging device inserted into the treatment site (such as a camera associated with the scope 32).

The catheter 48 can remove stone dust and fragments, wherein the fluid flow carries fluid and debris into the catheter 48 for removal therethrough. Generally, the debris may be cleared as it is generated (such as while the stone is being broken up). The removal of debris via the catheter 48 can take the place of the removal of fragments via ureteroscopic basketing, which can be relatively time consuming due to the difficulty of closing the basket around the stone, and due to the need to remove and re-insert the ureteroscope during each fragment removal. Therefore, using the catheter for stone removal can result in a more efficient removal procedure, which can provide the user greater confidence in removing an optimal amount of debris from the anatomy. Removing stone debris via the catheter can also reduce the risk of the stone fragment(s) injuring tissue compared to certain alternative stone removal methods, such as removal of stones through the ureter.

The catheter 48 can be used in several ways during a procedure. For example, the catheter 48 can be mobile throughout the procedure. The catheter can navigate around the treatment site to target specific stones or fragments in order to constrain them during lithotripsy, while also aspirating dust or debris. As another example, the catheter 48 can be initially stationary during the procedure and the scope 32 can be used to relocate stones to the catheter 48. The stones may be broken down at the catheter 48. At a later time during the procedure, the catheter 48 may be navigated through the treatment site to pick up remaining debris. As another example, the catheter 48 may be inserted (such as percutaneously) only when required, for example, during procedure escalation.

In some aspects, the point(s) of inflow (irrigation) may be separate from the point(s) of outflow (aspiration). For example, the inflow can be directed towards the point of outflow by deflecting the distal end of a first medical instrument (such as the endoscope 32) towards a second medical instrument (such as the percutaneous access instrument 40) such that the fluid flow is towards the second medical instrument. This may be accomplished robotically and/or manually with the systems and instruments described above with reference to FIG. 1. In some implementations, the point of outflow (aspiration) may be a single or concentrated point. More specifically, the point of outflow may be configured to provide high flow with high velocities so as to cause fragments to be pulled towards the point of outflow.

As described with reference to FIGS. 1 and 2, fluidics can be used for various purposes such as, for example, to achieve anatomical distension (such as for endoscopic vision), maintain suitable intrarenal pressures (such as to prevent damage to the anatomy), or move around objects (such as urinary stones) within the anatomy. Thus, the flow rates and/or pressures associated with irrigation and aspiration can affect various aspects of a medical procedure. In particular, fluid outflow is needed to prevent over-pressurization of the anatomy, which can result in fractures, tissue breakage, or damage to the anatomy. In some instances, fluid flowing out of the kidney 70 can adhere to the outer surface of the instrument 200 such that the fluid is exposed to the outside environment upon exiting the kidney 70. This fluid may flow a given distance along the outer surface of the instrument 200 before dropping off due to gravity. The falling waste fluid can pool or accumulate on the floor and/or other nearby surfaces, creating a hazardous environment for the physician and staff.

In some aspects, a fluid outflow control device may be placed around the outer surface of the instrument 200 to impede the flow of fluid along the outer surface and divert the fluid into a waste collection apparatus. Accordingly, the fluid outflow control device of the present disclosure can control the flow of fluid exiting the kidney 70 along the outer surface of instrument 200 to prevent such waste fluid from pooling or accumulating on the floor and/or other undesired surfaces. More specifically, the fluid outflow control device can ensure that most, if not all, of the waste fluid is diverted to and captured by a waste collection or management device so that the fluid can be properly measured, inspected, and/or disposed of. Among other advantages, the fluid outflow control device of the present disclosure can be coupled to the instrument 200 in a quick and robust manner, thereby reducing the workload (and cognitive load) on the user to ensure a clear and usable workspace and/or medical environment (such as by minimizing user distraction and/or maximizing user focus and attention). As a result, some medical procedures can be performed with fewer personnel (in the medical environment) and/or shorter durations using the fluid outflow control device.

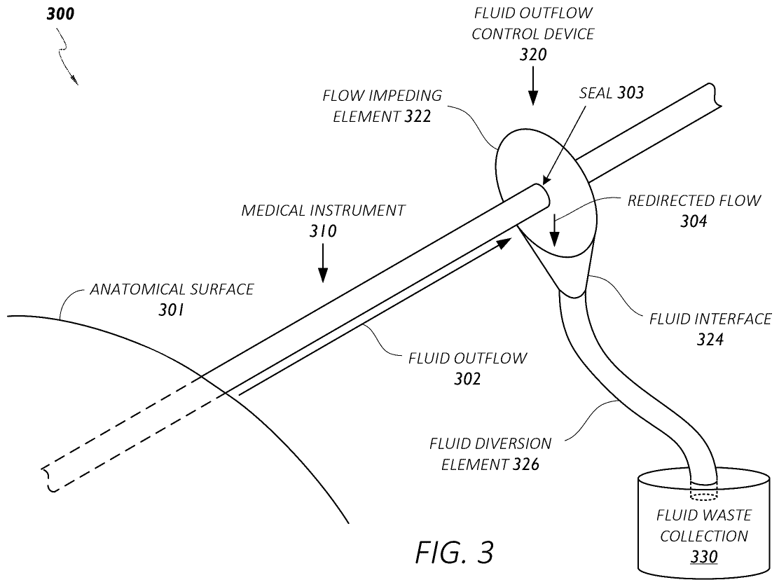

FIG. 3 shows an example medical system 300 including a fluid outflow control device 320, according to some implementations. The medical system 300 is shown to include a medical instrument 310 having an elongate shaft that is percutaneously inserted through an anatomical surface 301. For example, the anatomical surface 301 may represent the surface of a patient's skin or a target anatomy for percutaneous access (such as a kidney).

In some implementations, the medical instrument 310 may be one example of the catheter 48 of FIG. 1 or the medical instrument assembly 200 of FIG. 2. More specifically, the medical instrument 310 may provide passive and/or active aspiration to the target anatomy. For example, the proximal end of the medical instrument may be coupled to a vacuum or suction device (not shown for simplicity). In some implementations, irrigation also may be provided by the medial instrument 310 (such as shown in FIG. 2) or by another medical instrument (such as the direct-entry instrument 32 of FIGS. 1 and 2).

As shown in FIG. 3, at least some fluid flows out of the anatomy along an outer surface of the medical instrument 310. The direction of this fluid flow is depicted by a fluid outflow vector 302. The fluid outflow control device 320 is configured to be coupled to or disposed around the medical instrument 310 to catch the fluid flowing along the outer shaft and divert the fluid to a fluid waste collection apparatus 330. In some implementations, the fluid outflow control device 320 may include a flow impeding element 322, a fluid coupling interface 324, and a fluid diversion element 326. The flow impeding element 322 is configured to form a seal 303 around the outer surface of the elongate shaft that impedes the flow of fluid along the length of the shaft. More specifically, the seal 303 prevents fluid on the surface of the shaft from flowing past the flow impeding element 322 by redirecting at least some of the fluid onto an inner surface of the flow impeding element 322 (which faces the anatomical surface 301). This redirected fluid is pulled in the direction of gravity, as depicted by a redirected flow vector 304.

The flow impeding element 322 can be disposed around the medical instrument 310 at any point along the elongate shaft. For example, as shown in FIG. 3, the flow impeding element 322 may include a hole or opening (also referred to as a “receiving feature”) that allows the flow impeding element 322 to receive and/or mate with the medical instrument 310 to form the seal 303. In some implementations, the medical instrument 310 may be inserted through the receiving feature, so that the flow impeding element 322 can be slid to a desired position along the elongate shaft, before the medical instrument 310 is inserted into the anatomy. In some other implementations, the flow impeding element 322 may include one or more additional cuts and/or openings that provide lateral access to the receiving feature so that the flow impeding element 322 can be clamped (or clipped) onto the medical instrument 310, at any desired point along the elongate shaft, even after the medical instrument 310 has been inserted into the anatomy. As such, the flow impeding element 322 can be easily moved to different positions along the length of the shaft and/or to a new medical instrument to adjust for changes in the positioning of the shaft and/or changes in the medical procedure (such as where a new instrument is introduced).

The distance that the fluid can travel (unobstructed) along the length of the outer surface is governed by a number of factors, including the characteristics of the anatomical opening, the angle and/or depth of insertion of the medical instrument 310, the pressure and/or volume of the fluid, the force of gravity, surface characteristics of the medical instrument 310 (such as wettability), fluid composition, as well as cohesive and adhesive forces between the fluid and the outer surface of the medical instrument 310, among other examples. Many of these factors (such as the angle and depth of insertion of the medical instrument 310) can vary greatly across different medical procedures and/or over the course of the same procedure. As a result, the exact point at which the fluid will drop off from the medical instrument 310 (due to gravity) can be difficult to predict.

However, aspects of the present disclosure recognize that at least some of the factors affecting the distance that the fluid can travel along the length of the outer surface of the medical instrument 310 are relatively constant or stable (such as the surface chemistry of the elongate shaft). Thus, the fluid can be expected to travel at least a threshold (or minimum) distance along the surface of the shaft based on one or more known parameters. In some implementations, the flow impeding element 322 can be positioned within a threshold distance of the anatomical surface 301 to ensure that most, if not all, of the fluid flowing along the outer surface of the medical instrument is captured and redirected by the flow impeding element 322 before it would otherwise fall onto the floor and/or other surrounding surfaces. The positioning of the flow impeding element 322 along the shaft of the medical instrument 310 can be adjusted to accommodate varying depths of insertion by the medical instrument 310. As shown by the redirected flow vector 304, the flow impeding element 322 is configured to redirect the flow of fluid into the fluid diversion element 326.

The fluid diversion element 326 is configured to carry the fluid away from the flow impeding element 322 and deposit the fluid in the fluid waste collection apparatus 330. More specifically, the fluid diversion element 326 supports greater flexibility in the placement of the flow impeding element 322 and/or the fluid waste collection apparatus 330. For example, the flow impeding element 322 can be positioned closer to the anatomical surface 301 (such as to capture more fluid before it falls to the floor) and the fluid waste collection apparatus 330 can be positioned farther away from the anatomical surface 301 (such as to provide a less cluttered or more ergonomic operating environment). In some implementations, the fluid diversion element 326 may be a tubular structure that provides an enclosed fluid channel to prevent leakage or spilling of the fluid. To further reduce spilling, the terminal end of the fluid diversion element 326 can be clipped or otherwise disposed inside the fluid waste collection apparatus 330.

As shown in FIG. 3, the flow impeding element 322 has a larger surface area (or outer diameter) than the opening (or tubular cross-section) of the fluid diversion element 326. Having a larger surface area for the flow impeding element 322 helps prevent the redirected fluid from spilling over the edges of the flow impeding element 322 (such as when a large volume of fluid flows out of the anatomy at a high rate and/or pressure). On the other hand, having a smaller diameter of tubing for the fluid diversion element 326 allows for greater flexibility in routing the fluid between the flow impeding element 322 and the fluid waste collection element 330, while reducing or minimizing the interference or intrusiveness of the fluid diversion element 326 in the operating environment.

The fluid coupling interface 324 couples or connects the flow impeding element 322 to the fluid diversion element 326. More specifically, the fluid coupling interface 324 is configured to help guide fluid from the outer edge of the flow impeding element 322 into the opening of the fluid diversion element 326. In some implementations (such as where the outer diameter of the flow impeding element 322 is larger than the inner diameter of the fluid diversion element 326), the fluid coupling interface 324 may funnel the fluid from the flow impeding element 322 into the fluid diversion element 326. In other words, the fluid coupling interface 324 may ensure that the redirected fluid on the surface of the flow impeding element 322 is transferred into the fluid diversion element 326 without spilling or otherwise falling onto the floor or other undesired surfaces.

In the example of FIG. 3, the elements 322-326 of the fluid outflow control device 320 are shown to have specific shapes, features, and relative sizes. For example, the flow impeding element 322 is shown to have an annular structure with a substantially flat or planar surface, the fluid coupling interface 324 is shown to have a trapezoidal shape that tapers toward the fluid diversion element 326, and the fluid diversion element 326 is shown to bend or curve in the shape of an “S.” However, in actual implementations, the elements 322-326 of the fluid outflow control device 320 can have various other suitable shapes, sizes, dimensions, and/or geometries. In some implementations, one or more of the elements 322-326 of the fluid outflow control device 320 may be disposable and/or constructed from relatively inexpensive materials (such as rubber, plastic, or various other polymers). In some other implementations, one or more of the elements 322-326 of the fluid outflow control device 320 may be reusable and/or constructed from sturdier materials (such as steel or nitinol).

In some implementations, the elements 322-326 of the fluid outflow control device 320 may be discrete components that can be attached or coupled to one another to form the fluid outflow control device 320. Example suitable attachment mechanisms include adhesives, hook-and-loop fasteners, and press-fit connectors, among other examples. In such implementations, the elements 322-326 can be modular to create a fluid outflow control device 320 that is customized for a particular medical procedure. For example, the flow impeding element 322 of the fluid outflow control device 320 can be selected from a set of flow impeding elements of various geometries and/or sizes depending on the pressure and/or volume of fluid expected to flow out of the anatomy along the outer surface of the medical instrument 310. Similarly, the fluid diversion element 326 of the fluid outflow control device 320 can be selected from a set of fluid diversion elements 326 of various geometries and/or sizes depending on the distance between the flow impeding element 322 and the fluid waste collection apparatus 330. In some other implementations, two or more of the elements 322-326 of the fluid outflow control device 320 may be integrated with one another to form a single continuous object.

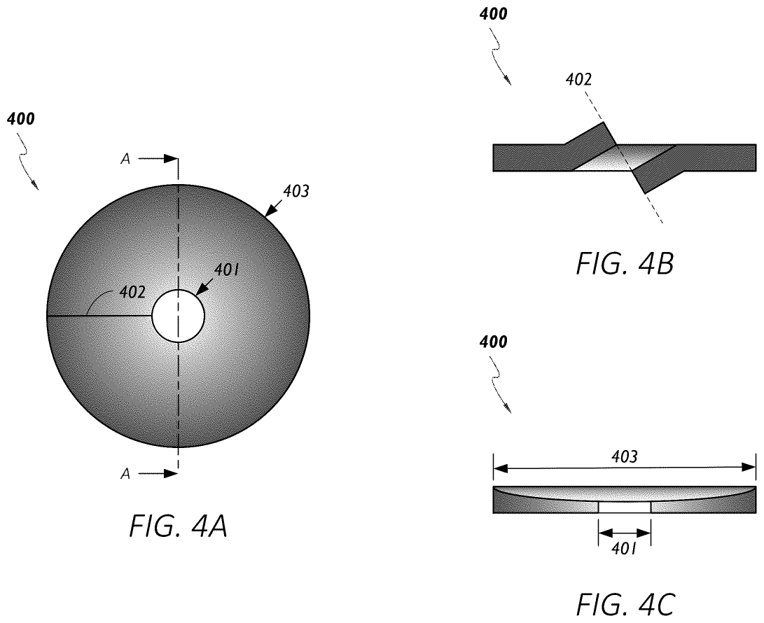

FIGS. 4A-4C shows an example flow impeding element 400 for a fluid outflow control device, according to some implementations. More specifically, FIG. 4A shows a top of view of the flow impeding element 400, FIG. 4B shows a side view of the flow impeding element 400, and FIG. 4C shows a sectional view of the flow impeding element 400 (representing the cross-section A-A shown in FIG. 4A).

In some implementations, the flow impeding element 400 may be one example of the flow impeding element 322 of FIG. 3. More specifically, the flow impeding element 400 is configured to block or otherwise impede a flow of fluid along the outer surface of an elongate shaft (such as the medical instrument 310) and redirect the flow of fluid onto an inner surface of the flow impeding element 400 (such as the surface facing an anatomy from which the fluid flows out onto the elongate shaft). In the example of FIGS. 4A-4C, the flow impeding element 400 has an annular structure (also referred to herein as an “annulus”) with an inner diameter 401 that forms an opening at the center of the annulus 400, and an outer diameter 403 that forms the outer edge or boundary of the annulus 400. The inner diameter 401 is configured to receive the elongate shaft and form a seal between the annulus 400 and the outer surface of the shaft (such as the seal 303 of FIG. 3) that can impede the flow of fluid along the outer surface of the shaft and redirect the fluid onto an inner surface of the annulus 400.

The annulus 400 has a radial cut 402 that extends from the inner diameter 401 to the outer diameter 403. The cut 402 breaks the continuity of the annulus 400 and allows the adjacent edges of the annulus 400 to be separated, as shown in FIG. 4B. More specifically, the cut 402 provides an opening between the inner diameter 401 and the outer diameter 403 so that the inner diameter 401 can be laterally accessed by the elongate shaft from a position tangential to the outer diameter 403. In other words, the cut 402 allows the annulus 400 to be clamped or clipped onto the elongate shaft (in addition to being slipped onto the elongate shaft via the inner diameter 401). In some implementations, the annulus 400 may be temporarily deformable so that the edges of the annulus 400 defined by the cut 402 can be stretched apart (such as shown in FIG. 4B) to receive the elongate shaft, and subsequently closed to return the annulus 400 to its original shape after the elongate shaft is disposed within the inner diameter 401.

In some implementations, the annulus 400 may be constructed or manufactured from deformable or elastic materials (such as rubber, plastic, or various other polymers) and the inner diameter 401 of the annulus may be slightly smaller than the outer diameter of the elongate shaft to form a tight seal around the surface of the shaft. In some other implementations, only a portion of the annulus 400 associated with the inner diameter 401 may be constructed from a deformable material while the remainder of the annulus 400 can be constructed using a more rigid or less compliant material (such as steel or nitinol). The materials used in constructing the annulus 400 may depend on various factors, including cost, reusability, weight, mechanism of placement, and compressibility or elasticity, among other factors. In some implementations, the annulus 400 may be a modular component of the fluid outflow control device. In other words, various annuluses can be constructed from different materials, having different shapes, sizes, dimensions, and/or geometries, and used interchangeably in the fluid outflow control device.

In the example of FIGS. 4A-4C, the inner surface of the annulus 400 is curved or otherwise angled (such as in the shape of a truncated cone) towards an anatomical surface (such as the anatomical surface 301 of FIG. 3) to catch or otherwise capture the fluid on the elongate shaft. In other words, the angle of the curvature brings the outer diameter 403 of the annulus 400 closer to the anatomical surface than the inner diameter 401 (similar to the form factor of a baseball glove, a bed pan, or a megaphone). The thickness of the annulus 400 is shown as a gradient, where thicker regions are depicted by darker shades of gray and thinner regions are depicted by lighter shades of gray. As shown in FIG. 4B, the outer diameter 403 of the annulus 400 is thicker than the inner diameter 401. This concave curvature of the inner surface helps funnel or direct the flow of fluid from the inner diameter 401 to the outer diameter 403 (such as along the direction of gravity). In some implementations, the inner surface of the annulus 400 may be textured to help direct the flow of fluid. In some other implementations, the inner surface of the annulus 400 may further include one or more channels that can direct the flow fluid from the inner diameter 401 to a single point on the outer diameter 403 of the annulus 400.

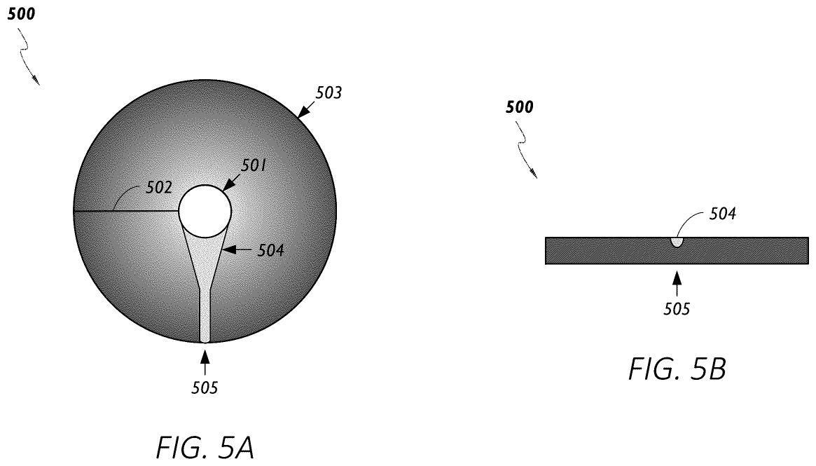

FIGS. 5A and 5B show another example flow impeding element 500 for a fluid control device, according to some implementations. More specifically, FIG. 5A shows a top view of the flow impeding element 500 and FIG. 5B shows a side view of the flow impeding element 500. The flow impeding element 500 is configured to block or otherwise impede a flow of fluid along the outer surface of an elongate shaft (such as the medical instrument 310 of FIG. 3) and redirect the flow of fluid onto an inner surface of the flow impeding element 500 (such as the surface facing an anatomy from which the fluid flows out onto the elongate shaft).

In some implementations, the flow impeding element 500 may be one example of the flow impeding element 400 of FIGS. 4A-4C. More specifically, the flow impeding element 500 is constructed as an annulus having an inner diameter 501, an outer diameter 503, and a radial cut 502 that extends from the inner diameter 501 to the outer diameter 503. As described with reference to FIGS. 4A-4C, the inner diameter 501 is configured to receive the elongate shaft and form a seal between the annulus 500 and the outer surface of the shaft (such as the seal 303 of FIG. 3) that can impede the flow of fluid along the outer surface of the shaft and redirect the fluid onto an inner surface of the annulus 500. The cut 502 provides an opening between the inner diameter 501 and the outer diameter 503 so that the inner diameter 501 can be laterally accessed by the elongate shaft from a position tangential to the outer diameter 503. The thickness of the annulus 500 is shown as a gradient, where thicker regions are depicted by darker shades of gray and thinner regions are depicted by lighter shades of gray.

In the example of FIGS. 5A and 5B, the inner surface of the annulus 500 further includes a channel 504 for directing a flow of fluid from the inner diameter 501 to a single point 505 along the outer diameter 502. More specifically, the channel 504 may control the flow of fluid along the inner surface of the annulus 500, rather than allow the flow of fluid to be dictated by various external factors (such as a decomposed gravitational force vector). As shown in FIG. 5B, the channel 504 may be cut or carved from the inner surface of the annulus 500 to guide the fluid along a desired path. As described with reference to FIG. 3, the channel 504 may help funnel the fluid into a fluid diversion element (such as the fluid diversion element 326) and prevent the fluid from spilling onto the floor or other undesired surfaces. In the example of FIGS. 5A and 5B, the channel 504 is shown in the shape of a funnel having a relatively shallow depth. However, in some other implementations, the channel 504 may have other suitable shapes, sizes, geometries, and/or depths.

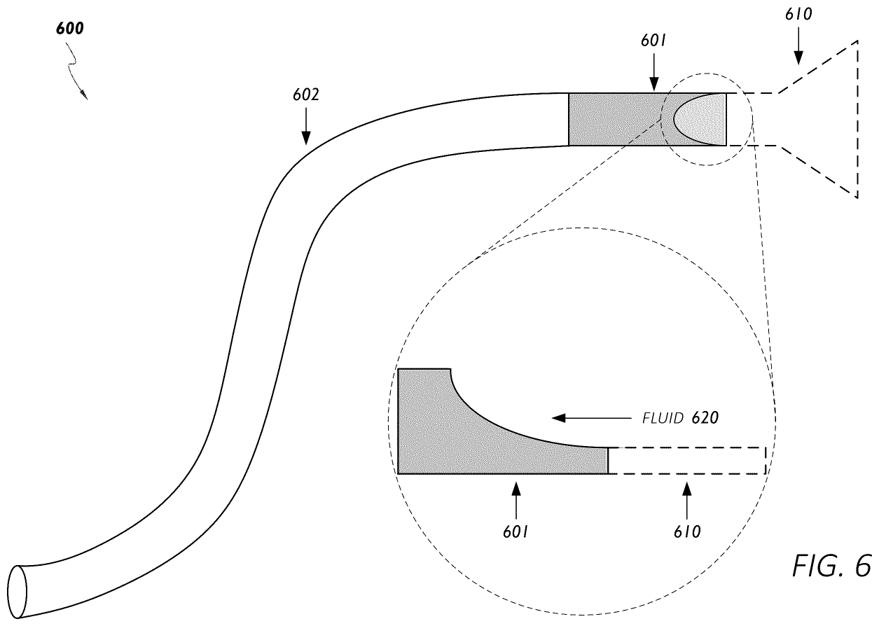

FIG. 6 shows an example fluid diversion element 600 for a fluid outflow control device, according to some implementations. The fluid diversion element 600 is configured to couple or attached to a flow impeding component of the fluid outflow control device (such as any of flow impending elements 322, 400, or 500 of FIGS. 3-5B). In some implementations, the fluid diversion element 600 may be one example of the fluid diversion element 326 of FIG. 3. More specifically, the fluid diversion element 600 is configured to carry fluid away from the flow impeding element and deposit the fluid in a waste collection apparatus (such as the fluid waste collection apparatus 330 of FIG. 3).

The fluid diversion element 600 has a tubular structure or “tubing” that provides an enclosed channel for transporting the fluid (such as to prevent leakage or spilling of the fluid during transport). The tube 600 includes a fluid intake component 601 and a fluid routing component 602. The fluid intake component 601 includes an opening to receive an inflow of fluid 620 from the flow impeding element. As shown by the exploded side view in FIG. 6, the opening of the fluid intake component 601 is curved or angled to avoid obstructing or otherwise interfering with the inflow of fluid 620. In some implementations, the fluid intake component 601 may be relatively stiff or rigid to ensure reliable funneling of the fluid 620 into the opening.

In some implementations, the fluid intake component 601 may include a funnel element 610 to help guide or funnel the inflow of fluid 620 from the flow impeding element into the opening of the fluid intake component 601. In some implementations, the funnel element 610 may be one example of the fluid coupling interface 324 of FIG. 3. For example, the funnel element 610 may attach or couple the fluid intake component 601 to the flow impeding element. The shape of the funnel element 610 also may provide a visual guide for attaching to the flow impeding element. In some implementations, the fluid intake component 601 may be even stiffer or more rigid than the fluid intake component 601. In some other implementations, the fluid intake component 601 may be configured to attach directly to the flow impeding component (in lieu of the funnel element 610).

The fluid routing component 602 is configured to route the fluid 620 to the waste collection apparatus. In some aspects, one or more portions of the fluid routing component 602 may be configured to deform, bend, flex, and/or expand (axially) to support various paths for routing the fluid 620 between the flow impeding element and the waste collection apparatus. In some implementations, one or more portions of the fluid routing component 602 may be constructed from tubing designed to geometrically deform or expand (such as accordion-type tubing or tubing in the shape of a coiled or “curly” cord). In some other implementations, one or more portions of the fluid routing component 602 may be constructed or manufactured from deformable or elastic materials having characteristic axial expansion properties (such as rubber).

The materials used in constructing the tubing 600 may depend on various factors, including cost, reusability, weight, mechanism of placement, and compressibility or elasticity, among other factors. In some implementations, the stiffness or rigidity of the tubing 600 may vary along its length. For example, the intake component 601 may be stiffer or more rigid than the fluid routing component 602. In some implementations, the tubing 600 may be a modular component of the fluid outflow control device. In other words, various tubing can be constructed from different materials, having different lengths, stiffness, geometries, and/or other dimensions, and used interchangeably in the fluid outflow control device.

In some implementations, the tubing 600 may further include a clip or other attachment mechanism (not shown for simplicity) for securing the terminal end of the tubing 600 (opposite the intake component 601) to the waste collection apparatus. The tubing 600 may support a variety of attachment mechanisms associated with various catchment or collection systems. Example suitable attachment mechanisms include clips, adhesives, and magnets, among other examples. The attachment mechanism can further help control an outflow of the fluid 620 from the terminal end of the tubing 600 to ensure that the fluid 620 is securely deposited within the waste containment apparatus (such as to avoid spills or leaks). For example, by clipping the terminal end of the tubing 600 at least partially inside the waste collection apparatus (or catchment bag), aspects of the present disclosure can prevent the fluid 620 from spilling onto the floor and/or other undesired surfaces upon exiting the tubing 600.

In the foregoing specification, implementations have been described with reference to specific examples thereof. It will, however, be evident that various modifications and changes may be made thereto without departing from the broader scope of the disclosure as set forth in the appended claims. The specification and drawings are, accordingly, to be regarded in an illustrative sense rather than a restrictive sense.

As used herein, a phrase referring to “at least one of” a list of items refers to any combination of those items, including single members. As an example, “at least one of: a, b, or c” is intended to cover: a, b, c, a-b, a-c, b-c, and a-b-c.

Various modifications to the implementations described in this disclosure may be readily apparent to those skilled in the art, and the generic principles defined herein may be applied to other implementations without departing from the spirit or scope of this disclosure. Thus, the claims are not intended to be limited to the implementations shown herein, but are to be accorded the widest scope consistent with this disclosure, the principles and the novel features disclosed herein.

What is claimed is:

1. A medical system, comprising:
a medical instrument having an elongate shaft configured to be inserted percutaneously into an anatomy;
a collection apparatus configured to capture fluid that flows along an outer surface of the elongate shaft; and
an outflow control device configured to impede the flow of fluid along the outer surface of the elongate shaft and divert the fluid to the collection apparatus.

2. The medical system of claim 1, wherein the medical instrument is configured to provide aspiration of fluid within the anatomy.

3. The medical system of claim 1, wherein the outflow control device comprises:
a flow impeding element configured to form a seal around the outer surface of the elongate shaft that prevents the fluid on the outer surface from flowing beyond the flow impeding element.

4. The medical system of claim 3, wherein the flow impeding element comprises an annular structure having an inner opening that conforms to the outer surface of the elongate shaft to form the seal around the outer surface.

5. The medical system of claim 4, wherein at least the inner opening of the annular structure comprises a deformable or elastic material.

6. The medical system of claim 4, wherein the annular structure includes a channel for guiding the fluid from the inner opening to an outer edge of the annular structure.

7. The medical system of claim 3, wherein the seal further causes the fluid to flow from the outer surface of the elongate shaft onto a surface of the flow impeding element.

8. The medical system of claim 7, wherein the outflow control device further comprises:
a fluid diversion element configured to form a channel for carrying the fluid between the flow impeding element and the collection apparatus.

9. The medical system of claim 8, wherein the fluid flows from the flow impeding element to the fluid diversion element, at least in part, due to gravity.

10. The medical system of claim 8, wherein the fluid diversion element comprises one or more tubular structures.

11. The medical system of claim 8, wherein a rigidity of the fluid diversion element varies along a length of the channel.

12. The medical system of claim 8, wherein the outflow control device further comprises:
an interface element configured to funnel the fluid from the surface of the flow impeding element into the fluid diversion element.

13. A fluid outflow control device for a medical instrument having an elongate shaft, the fluid outflow control device comprising:
a flow impeding element configured to form a seal around an outer surface of the elongate shaft that redirects a flow of fluid along the outer surface onto a surface of the flow impeding element;
a fluid diversion element configured to form a channel for carrying the fluid between the flow impeding element and a collection apparatus; and
an interface element configured to funnel the fluid from the surface of the flow impeding element into the fluid diversion element.

14. The fluid outflow control device of claim 13, wherein the seal further prevents the fluid on the outer surface of the elongate shaft from flowing beyond the flow impeding element.

15. The fluid outflow control device of claim 13, wherein the flow impeding element comprises an annular structure having an inner opening that conforms to the outer surface of the elongate shaft to form the seal around the outer surface.

16. The fluid outflow control device of claim 15, wherein at least the inner opening of the annular structure comprises a deformable or elastic material.

17. The fluid outflow control device of claim 15, wherein the annular structure includes a channel for guiding the fluid from the inner opening to an outer edge of the annular structure.

18. The fluid outflow control device of claim 13, wherein the fluid flows from the flow impeding element to the fluid diversion element, at least in part, due to gravity.

19. The fluid outflow control device of claim 13, wherein the fluid diversion element comprises one or more tubular structures.

20. The fluid outflow control device of claim 13, wherein a rigidity of the fluid diversion element varies along a length of the channel.
